(19) United States
(12) Patent Application Publication
(10) Pub. No.: US 2005/0187433 A1
(43) Pub. Date: Aug. 25, 2005

(54) In-vivo imaging device providing constant bit rate transmission

(75) Inventors: Eli Horn, Kiryat Motzkin (IL); Ofra Zinaty, Haifa (IL)

Correspondence Address: PEARL COHEN ZEDEK, LLP, 10 ROCKEFELLER PLAZA, SUITE 1001, NEW YORK, NY, 10020, US

(73) Assignee: GIVEN IMAGING LTD.

(21) Appl. No.: 11/087,606

(22) Filed: Mar. 24, 2005

Related U.S. Application Data
(63) Continuation-in-part of application No. 10/991,098, filed on Nov. 18, 2004.
Application No. 10/991,098 is a continuation-in-part of application No. 10/202,626, filed on Jul. 25, 2002, now abandoned.
(60) Provisional application No. 60/307,605, filed on Jul. 26, 2001.

(51) Int. Cl. A61B 1/06

(52) U.S. Cl. 600/160

(57) ABSTRACT

A device, system and method may enable the obtaining of in-vivo images or other data from within body lumens or cavities, such as images of the gastrointestinal (GI) tract, where the data obtained at a variable bit rate may be transmitted at a constant bit rate. An in-vivo buffer or storage device may be used to store data prior to transmission.

### CROSS-REFERENCE TO RELATED APPLICATION

This application is a continuation-in-part of U.S. patent application Ser. No. 10/991,098, filed Nov. 18, 2004, entitled “Diagnostic Device Using Data Compression”, which in turn is a continuation-in-part of U.S. patent application Ser. No. 10/202,626 filed on Jul. 25, 2002, which in turn claims benefit from prior provisional application No. 60/307,605 entitled “Imaging Device Using Data Compression” and filed on Jul. 26, 2001 all of which are incorporated by reference herein in their entirety.

### FIELD OF THE INVENTION

The present invention relates to an in-vivo device, system, and method for transmitting data from within a body lumen.

### BACKGROUND OF THE INVENTION

Devices, systems, and methods for performing in-vivo imaging, for example, of passages or cavities within a body, and for gathering information other than or in addition to image information (e.g. temperature information, pressure information, etc.), are known in the art. Such devices may include, inter alia, various endoscopic imaging systems and various autonomous imaging devices for performing imaging in various internal body cavities.

An in-vivo imaging device may, for example, obtain images from inside a body cavity or lumen, such as the gastrointestinal (GI) tract. An external receiver/recorder, for example, worn by a patient, may record and store images and other data. Images and other data may be displayed and/or analyzed on a computer or workstation after downloading the data recorded. Transmission may be wireless, for example, by RF communication with constant rate transmission or via wire.

Image data and/or other data may be compressed prior to transmission. Methods for lossy and lossless compressing image or video data may be known, for example, compression algorithms such as JPEG and MPEG may be used to compress image and video data. The size of the compressed image data may be variable and may depend on the content of data within the image(s) being compressed.

### SUMMARY OF THE INVENTION

An embodiment of the device, system and method of the present invention may enable obtaining transmission of in-vivo image data from within body lumens or cavities, such as images of the gastrointestinal tract, via constant rate transmission. In some embodiments of the present invention a buffer or other data storage unit may be used to temporarily store data obtained at a variable bit rate before transmission. In other embodiments of the present invention, the occupancy or fullness level of the buffer may be controlled by altering the mode of operation or otherwise altering the operation of the in-vivo device.

### BRIEF DESCRIPTION OF THE DRAWINGS

The present invention will be understood and appreciated more fully from the following detailed description taken in conjunction with the drawings in which:

FIG. 1 shows a schematic diagram of an in-vivo imaging system according to embodiments of the present invention;

FIG. 2A shows an exemplary mosaic pixel arrangement according to embodiments of the present invention;

FIG. 2B shows an exemplary red pixel plane of a mosaic pixel arrangement according to embodiments of the present invention;

FIG. 2C shows an exemplary blue pixel plane of a mosaic pixel arrangement according to embodiments of the present invention;

FIG. 2D shows an exemplary first green pixel plane of a mosaic pixel arrangement according embodiments of the present invention;

FIG. 2E shows an exemplary second green pixel plane of a mosaic pixel arrangement according to embodiments of the present invention;

FIG. 3 shows a flow chart describing a method of compressing image data according to an embodiment of the present invention;

FIG. 4 shows a transformation of mosaic data pixel arrangement from an [R, G1, G2, B] color space to an alternate color space according to an embodiment of the present invention;

FIG. 5 shows a flow chart describing a method for controlling data flow through a buffer according to an embodiment of the present invention;

FIG. 6 shows a method for compressing image data according to a defined mode in accordance with an embodiment of the present invention;

FIG. 7 shows a flow chart describing a method for defining a mode of compression to control the data flow through a buffer according to an embodiment of the present invention;

FIG. 8 shows a flow chart describing a method for decompressing data according to an embodiment of the present invention; and

FIG. 9 shows a flow chart describing a method for decompressing data that was compressed with a defined mode according to an embodiment of the present invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Embodiments of the device, system and method of the present invention may be used, for example, in conjunction with an imaging system or device such as may be described in U.S. Pat. No. 5,604,531 to Iddan et al. and/or in U.S. application Ser. No. 09/800470 entitled “A Device And System For In Vivo Imaging”, published as application No. 20010035902, both of which are hereby incorporated by reference However, the device, system and method according to the present invention may be used with any suitable device that may provide image and/or other data from within a body lumen or cavity. In alternate embodiments, the system and method of the present invention may be used with devices capturing information other than image information within the human body; for example, temperature, pressure or pH information, information on the location of the transmitting device, or other information.

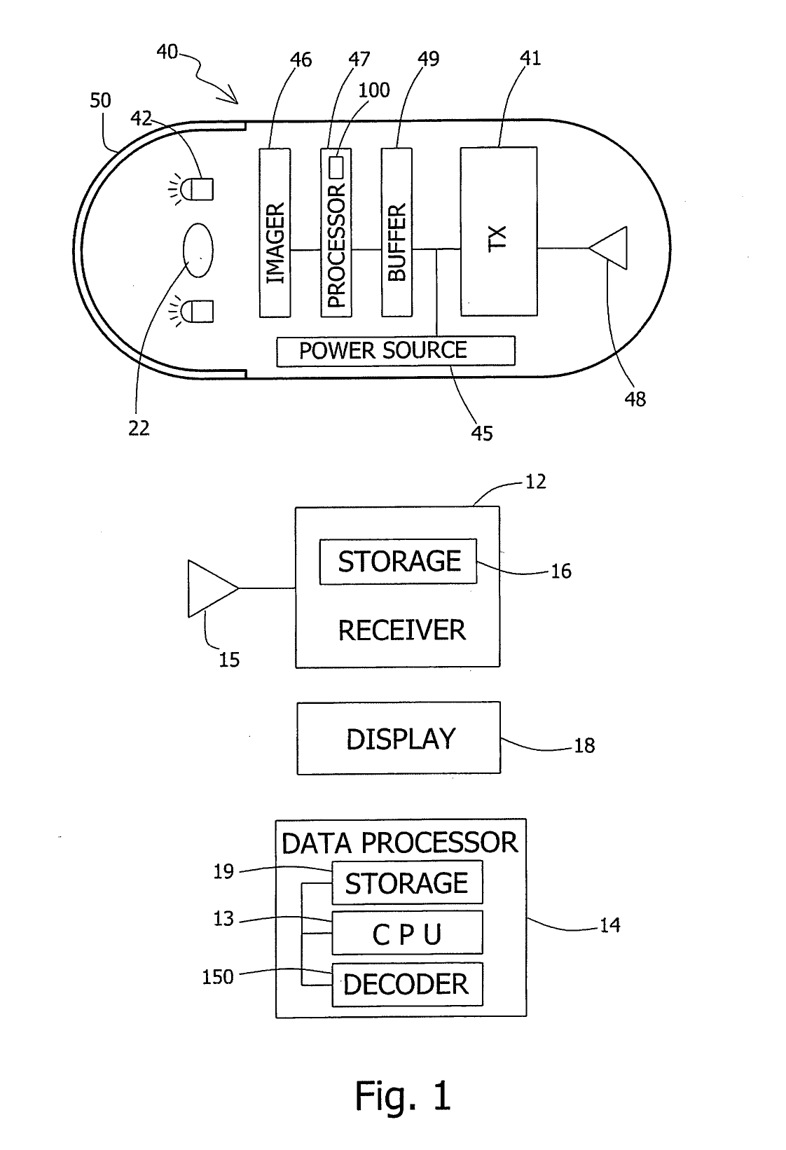

Reference is made to FIG. 1, showing a schematic diagram of an in-vivo imaging system according to embodiments of the present invention. In an exemplary embodiment, a device 40 may be a swallowable capsule capturing images, for example, images of the gastrointestinal tract. Device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power or transmit information. Device 40 may communicate with an external receiving and display system to provide display of data, control, or other functions. For example, power may be provided by an internal battery or a wireless receiving system. Other embodiments may have other configurations and capabilities. For example, components may be distributed over multiple sites or units. Control information may be received from an external source.

Typically, device 40 may be an autonomous device and may include at least one sensor such as an imager 46, for capturing image frames, a viewing window 50, a processing chip or circuit 47 that may process signals generated by the imager 46, one or more illumination sources 42, an optical system 22, a transmitter 41 and a power source 45, for example a battery. In one embodiment of the present invention, the imager 46 may be and/or contain a CMOS imager. In other embodiments, other imagers may be used, e.g. a CCD imager or other imagers. Processor 47 and or imager 46 may incorporate circuitry, firmware, and/or software for compressing images and/or other data, e.g. control data. In other embodiments, a compression module 100 and/or a buffer 49 or other suitable storage unit (e.g., a memory, one or more registers, etc.) may be incorporated in the imager 46 or a processor 47, e.g., a processing chip or other suitable processor, transmitter 41, and/or a separate component. In some embodiments of the present invention, the buffer 49 may have a capacity that may be substantially smaller than the size of a frame of compressed and/or non-compressed image data captured, for example, from imager 46. Processor 47 need not be a separate component; for example, processing circuitry 47 or its functionality may be integral to the imager 46, integral to a transmitter 41 and/or other suitable components of device 40. The buffer 49 may, for example, have a capacity of 0-20 kilobytes, e.g. 3 kilobytes, and may serve to facilitate, for example, a constant bit rate of data from the compression module 100 to the transmitter 41, for example, a transmission rate of 1-10 megabits/second, e.g 5 megabits/second. Other uses of buffer 49 and/or other sizes of buffer 49 may be implemented. The transmitter 41 may, for example, transmit compressed data, for example, compressed image data and possibly other information (e.g., control information) to a receiving device, for example a receiver 12. In other embodiments, uncompressed data may be transmitted. The transmitter 41 may typically be an ultra low power radio frequency (RF) transmitter with high bandwidth input, possibly provided in chip scale packaging. The transmitter may transmit, for example, via an antenna 48. The transmitter 41 may, for example, include circuitry and functionality for controlling the device 40.

Typically, device 40 may be an autonomous wireless device that may be, for example, swallowed by a patient and may traverse a patient's GI tract. However, other body lumens or cavities may be imaged or examined with device 40. Device 40 may transmit image and possibly other data in a compressed format to, for example, components located outside the patient's body, which may, for example, receive and process, e.g. decode, the transmitted data. Data may be transmitted in an uncompressed format. According to one embodiment, located outside the patient's body in one or more locations, may be a receiver 12, preferably including an antenna or antenna array 15, for receiving image and possibly other data from device 40, a receiver storage unit 16, for storing image and other data, a data processor 14 with CPU 13, a data processor storage unit 19, a data decoding module 150 for decompressing data, and an image monitor and/or display 18, for displaying, inter alia, the images transmitted by the device 40 and recorded by the receiver 12. In one embodiment of the present invention, receiver 12 may be small and portable. In other embodiments receiver 12 may be integral to data processor 14 and antenna array 15 may be electrically communicated to receiver 12 by, for example, wireless connections. Other suitable configurations of receiver 12, antenna array 15 and data processor 14 may be used. Preferably, data processor 14, data processor storage unit 19 and monitor 18 may be part of a personal computer, workstation, or a Personal Digital Assistant (PDA) device, or a device substantially similar to a PDA device. In alternate embodiments, the data reception and storage components may be of other suitable configurations. Further, image and other data may be received in other suitable manners and by other sets of suitable components.

In-vivo autonomous devices, for example, device 40, may typically have limited space and power provision and therefore it may be desirable to minimize the processing power and buffer size and/or memory that may be required for compressing data. Other design considerations may govern size, data capacity, processing capacity, and other specifications. In some embodiments of the present invention, it may be desirable to accomplish compression of image and other data without memory capability.

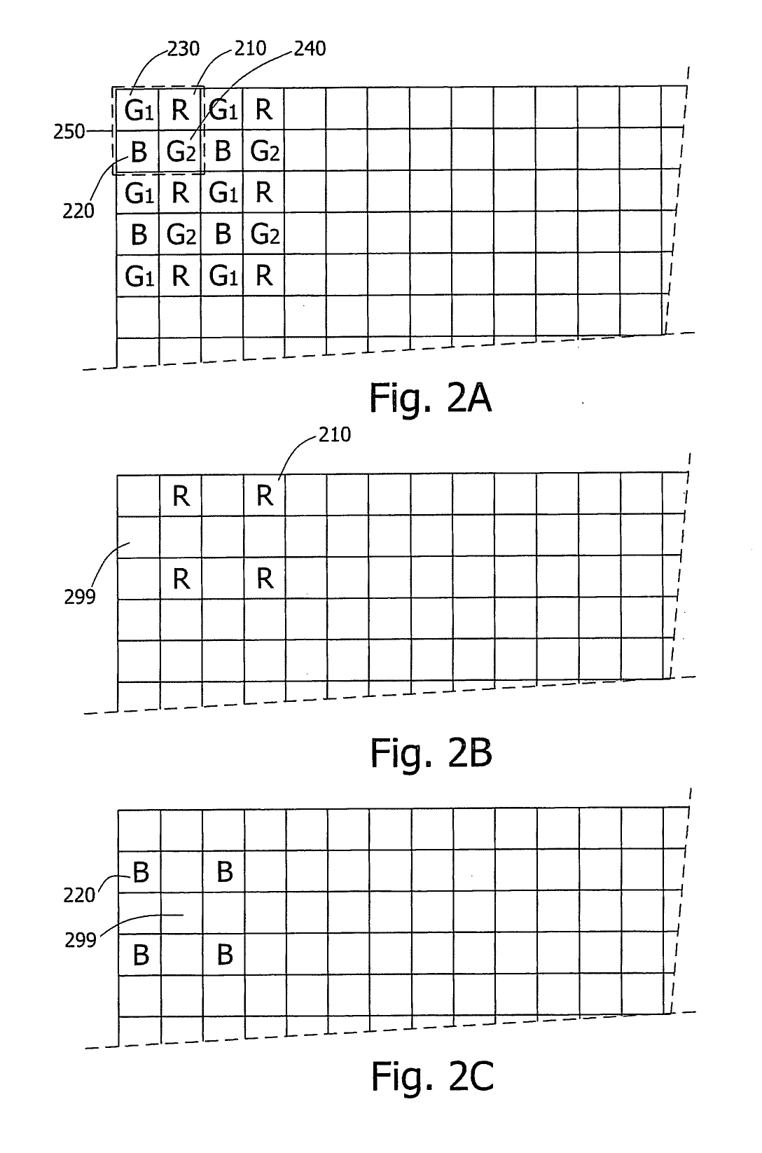

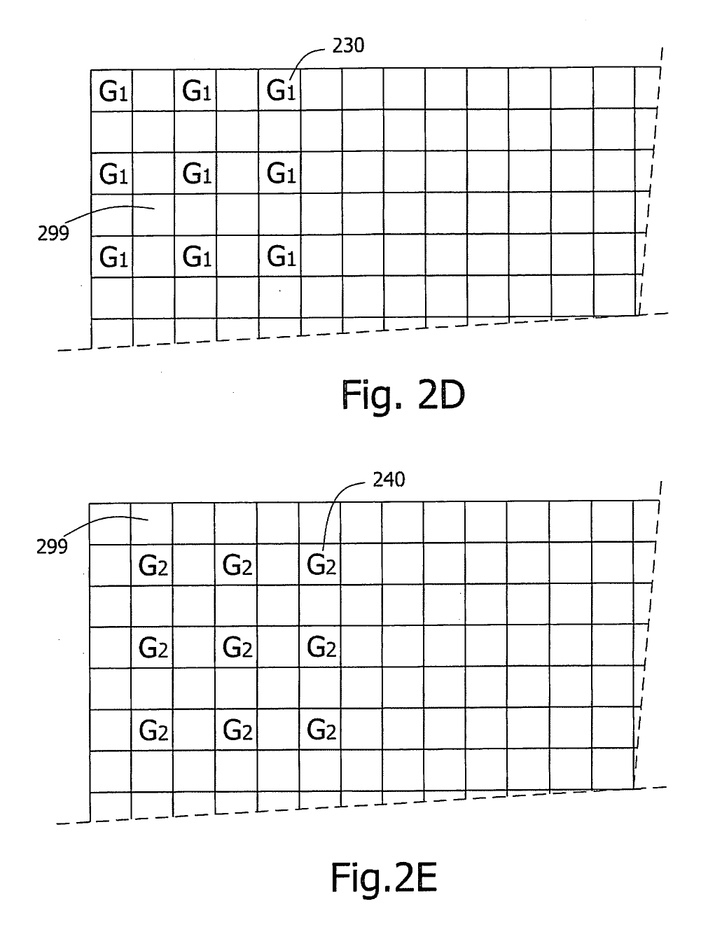

Reference is now made to FIG. 2 showing an exemplary mosaic pixel arrangement according to an embodiment of the present invention. In FIG. 2 every pixel group 250 may be represented by a mosaic of four pixels, for example, a red pixel 210, a blue pixel 220, a first green pixel 230, and a second green pixel 240. Corresponding pixel planes; red, blue, first green and second green planes according to the pixel arrangement shown in FIG. 2A are shown in FIGS. 2B, 2C, 2D and 2E respectively. Known methods, e.g. known interpolation methods, may be used to create a complete Red, Green and Blue (RGB) pixel image that may include an RGB value in each of the pixel positions 299. Other suitable pixel arrangements are possible, and color data need not be used.

The data compression module 100 and decoder module 150 may use various suitable data compression methods and systems. The data compression methods used may be lossless or lossy. Lossless data compression may enable precise (typically with no distortion) decoding of the compressed data. The compression ratio of lossless methods may however be limited. Lossy compression methods may not enable precise decoding of the compressed information. However the compression ratio of lossy methods may typically be much higher than that of lossless methods, for example lossy compression may result in compression ratios greater than two. In many cases the data distortion of lossy methods may be non-significant and/or non-discernable with the human eye. Typically, known compression algorithms may compress or decrease the original size of the data by storing and/or transmitting only differences between one or more neighboring values, for example, pixels. In general, differences in color and intensity of images data may typically occur gradually over a number of pixels, and therefore the difference between neighboring pixels may be a smaller quantity as compared to the value of each pixel. In some embodiments of the present invention, a compression ratio of at least four may be desired.

Known compression algorithms may include JPEG and/or MPEG compression that may typically be used for image and video compression and may have an option to operate according to either a lossless or lossy scheme. However such algorithms may typically require relatively substantial processing power and memory, and may typically require full RGB data (as opposed to mosaic data) as input. Other known compression algorithms (that may typically be lossless), e.g., Binary Tree Predictive Coding (BTPC), Fast Efficient Lossless Image Compression System (FELICS), and Low Complexity Context-Based Lossless Image Compression Algorithm (LOCO-I), etc., may require relatively lower processing power and memory but may still require memory to, for example store tables and other data. In some embodiments of the present invention, known lossy and/or lossless methods may be revised and/or implemented with pre-processing of data to enable lossy compression to be implemented with reduced processing power and memory requirements, and to be compatible with mosaic image data input.

In some embodiments of the present invention, the performance of known compression algorithms may be improved by considering known characteristics of the system and the environment that may be imaged. In one example, the resolution of an image may be limited by, for example, optical system 22. Knowledge of the known limitations in the resolution may be, for example, incorporated into compression algorithms, e.g. by defining one or more parameters, so that higher performance may be achieved for the specific system being used. In other embodiments, a priori knowledge of the color scheme, or other characteristics of the image data may be incorporated in the compression algorithm. In other embodiments pre-processing may be implemented to increase performance of the compression algorithm. Performance may be based on compression ratio, processing, and/or memory requirement. Embodiments of the present invention describe a system and method for compression of image and other data at high compression rates, e.g. compression ratios of 4-8, with substantially little processing power and memory requirements.

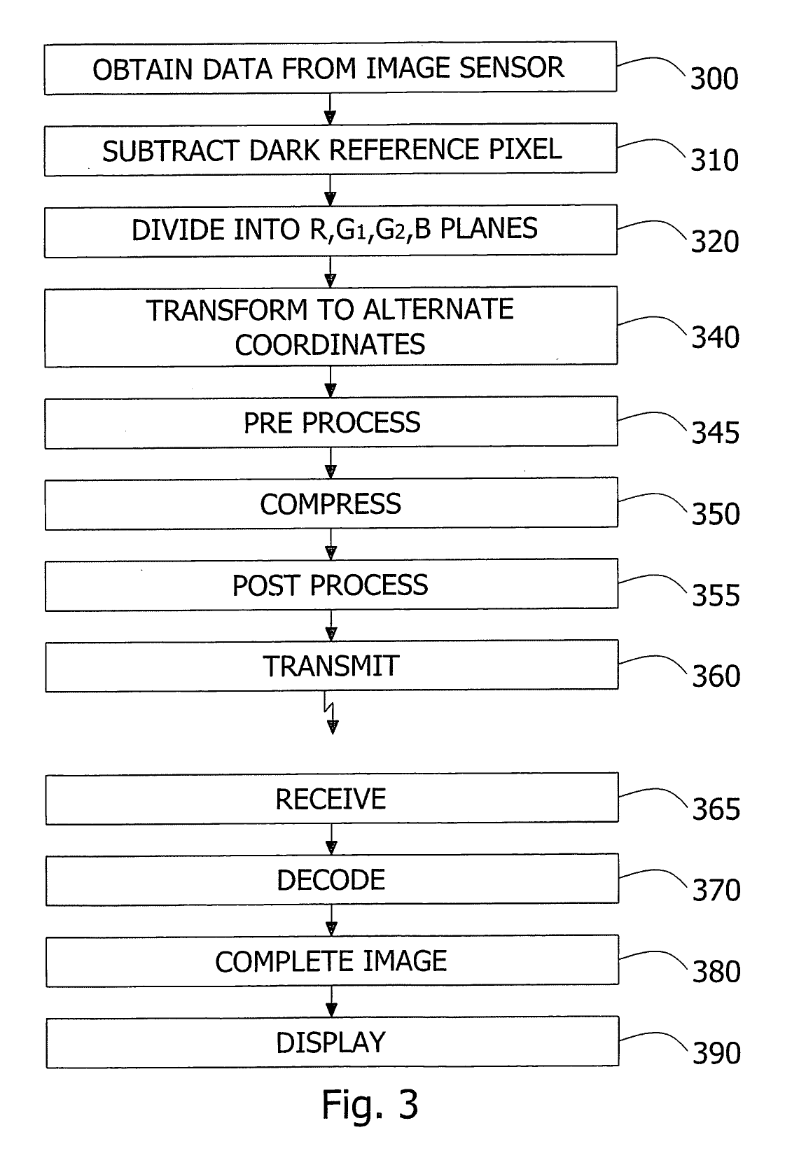

Reference is now made to FIG. 3 showing a method for compressing data according to one embodiment of the present invention. In block 300 image data may be obtained. In other embodiments data other than and/or in addition to image data may be obtained and compressed. Image data 300 may be mosaic image data, for example, as may be shown in FIG. 2, complete RGB color image data, or other suitable formats of image data. Typically, for in-vivo devices, image data may be mosaic image data as may be described herein. In block 310 dark reference pixels may be subtracted from corresponding pixels. In other embodiments of the present invention, dark reference pixels may not be provided for each captured image. In some embodiments of the present invention, dark image information may be obtained using other known methods. For example, a dark image may be captured for a plurality of captured images. Subtraction of the dark image may be performed during or post decoding of the captured image using suitable methods. Dark images may be, for example, interpolated using suitable methods to estimate the dark image noise corresponding to each of the captured images. In some embodiments of the present invention, compression and/or processing of data may result in shifting or distortion of image data. Compressing the dark images using similar steps and parameters as compression of the captured images may maintain the correspondence between the captured and dark images so that proper subtraction of dark image noise may be achieved. In block 320, the pixel plane may be divided, for example, into mono-color pixel planes, for example, planes shown in FIG. 2B-2E. In one embodiment of the present invention, compression algorithms, for example, known predictive types of compression, may be performed on each of the mono-color pixel planes after filling the missing information on each of the planes. Other suitable compression algorithms besides known predictive type algorithms may be implemented. In some embodiments of the present invention, the mono-color plane may be filled using known algorithms, e.g. known interpolation methods to obtain data corresponding to a full RGB image. In some embodiments, compression may be performed directly on the mosaic plane (FIG. 2A) or the mono-color planes (FIG. 2B-2E) without completing the RGB image. Known image compression algorithms, for example JPEG, may require a full RGB image before compression data. When applying this algorithm to mosaic image data, compression may require extra processing to fill and/or complete missing data as well as extra storing capability to store the larger size data. In some embodiments of the present invention, a compression method for directly compressing mosaic image data, e.g. data that is not full RGB data, without requiring a complete or substantially complete RGB image may be provided. Neighboring pixels required for pixel comparison in the compression block may be defined and/or located (e.g. with pointers) as the closest pixels available having the same color. In block 340, a transformation may be performed to transform, for example, the RGB plane and/or coordinates (or [R, G1, G2, B] coordinates) of the image into alternate coordinates, according to embodiments of the present invention. Sample coordinates and/or dimensions may include, for example, Hue, Saturation, and Value [H, S, V] coordinates, [Y, I, Q] planes commonly used for television monitors, or [Y, U, V] commonly used in JPEG compression. In other embodiments, other dimensions and/or coordinates suitable for images captured in-vivo may be used, e.g. images of in-vivo tissues. In some embodiments of the invention, data may be a combination of image and non-image data, for example one or more dimensions of the data may represent non-image data.

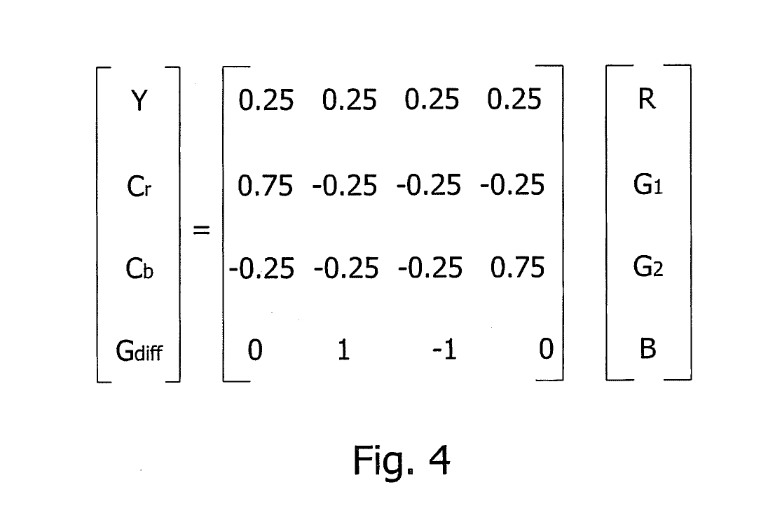

Reference is now made to FIG. 4 showing an exemplary transformation according to an embodiment of the present invention using, for example, four coordinates, e.g. [Y, Cr, Cb, Gdiff], corresponding to, for example, the four pixels in the pixel group 250 shown in FIG. 2A. Other coordinates, transformations, and defined pixels and/or pixels groups may be used. Y may be, for example, representative of the intensity of an image, Cr may be, for example, representative of the color red, Cb may be, for example, representative of the color blue, and Gdiff may be, for example, representative of the difference between the first and second green pixels. Other transformations and/or other coordinates may be used in other embodiments of the present invention. In some embodiments of the invention, subtraction of dark reference pixels may be performed after transformation to alternate coordinates. Referring back to FIG. 3, pre-processing of data (block 345) may be performed, for example, one or more of the dimensions and/or coordinates may be discarded. Discarding a dimension may enable, for example, simplification of the computations subsequently required and/or reduction in the quantity of data to be handled and transmitted, e.g. increase the compression ratio. In block 350, a compression algorithm may be implemented. In some embodiments of the present invention, compression may be performed “on the fly”, for example, in units of a few lines at a time, e.g. four lines of pixels. In other examples, compression “on the fly” may be performed with more or less than four lines of pixels. Typically compression performed by compression module 100 may be based on a variety of known predictive codes, e.g. (BTPC, FELICS, LOCO-I) that may typically use information from, for example, two or more neighboring pixel, e.g. a pixel above and the pixel to the left. Other suitable methods of lossless and/or lossy compression may be implemented. To increase the performance of the compression algorithm used, e.g. increase the compression ratio; pre-processing (block 345) and/or post processing (block 355) of the data may be performed. For example, one or more Least Significant Bits (LSBs) in one or more dimension and/or parameters of the data may be discarded, e.g. during pre-processing, to increase the compression ratio in one or more dimensions of the data. In other examples, a priori knowledge of, for example, typical color may be implemented to, for example, emphasis and/or de-emphasis particular dimensions of an image. In yet other examples, a priori knowledge of the characteristics of the typical object imaged may be implemented to emphasis and/or de-emphasis details or local changes that may be known to or not to typically occur. For example, for imaging of in-vivo tissue, certain sharp changes in color may not be typical and if encountered in image data may, for example, be de-emphasized. In still yet other examples, a priori knowledge of the particular optical system used may be implemented to emphasize or de-emphasize details encountered. For example, if a sharp detail may be encountered and the optical system, for example optical system 22, may be known not to provide the capacity to discern such sharp details, this detail may be de-emphasized by for example discarding the LSB, performing smoothing, decimation, etc. Emphasis and/or de-emphasis may be implemented by, for example, pre-processing (prior to compression), post-processing, and/or defining parameters of the compression algorithm. In one embodiment of the present invention, one or more dimension of the image may, for example, be discarded. For example, if one or more of the dimension may be known to produce a relatively flat image in the environment being imaged, that dimension may, for example, be discarded and no computations may be performed in that dimension. This may serve to, for example, increase the compression ratio, decrease the memory required to perform compression, and simplify the coding so that less processing may be needed. In other embodiments of the present invention, processing required as well as the memory required for the coding may be reduced by, for example, customizing known algorithms, for example, by eliminating the adaptive part of the coding that may require large tables of accumulated data. By implemented a priori knowledge of typical images and/or data that may normally be captured with, for example, a particular sensing device capturing images, for example, in a particular environment, the adaptive part of the coding may be reduced to a minimum. Data other or in addition to image data may be compressed.

In block 360, compressed data may be transmitted. Compressed data as well as encoded data and/or control data may be transmitted, for example in a compressed format. In other embodiments, encoded data and/or control data may not be compressed. Due to compression or other factors each line may have a variable bit length. In some embodiments of the present invention, a buffer 49 or other suitable storage unit may stall or hold data until, for example, a predetermined quantity of data may be accumulated for transmission, a predetermined time or number of frames has passed, etc. In some embodiments of the present invention, a buffer 49 may temporarily stall data to adapt the output from the compression that may have a variable bit rate to a constant bit rate transmission. Transmission may be in portions substantially smaller than an image frame, for example, a portion may be one or more lines of data, e.g. 270 bits. In one embodiment, entire images need not be stalled and/or stored. Data transmitted may be received (block 365) stored and subsequently decoded (block 370). In other embodiments of the present invention, decoding may be performed “on the fly” directly upon transmission. If dark reference pixels have not been subtracted during image capture, dark image information may be subtracted, for example, post decoding. The decoded mosaic image may be completed (block 380), e.g., by using known methods, e.g. known interpolation methods. The image may be displayed (390).

Typically, the data compression module 100 and decompression module 150 may include circuitry and/or software to perform data compression. For example, if the data compression module 100 and/or decompression module 150 may be implemented as a computer on a chip or ASIC, data compression module 100 or decompression module 150 may include a processor operating on firmware which includes instructions for a data compression algorithm. If data decompression module 150 may be implemented as part of data processor 14 and/or CPU 13, the decompression may be implemented as part of a software program. Other suitable methods or elements may be implemented.

In some embodiments of the present invention, the rate of transmission may be, for example, approximately 5.41 Megabits per second. Other suitable rates may be implemented. Compression may reduce the rate of transmission required or increase the quantity of information that may be transmitted at this rate. According to some embodiments, randomization may be implemented (performed, for example, by the transmitter 41). Namely, the occurrence of the digital signals (“0” and “1”) may be randomized so that transmission may not, for example, be impeded by a recurring signal of one type. In some embodiments of the present invention, an Error Correction Code (ECC) may be implemented before transmission of data to protect the data transmitted, for example, Bose Chaudhuri Hocquenghem (BCH) may be used.

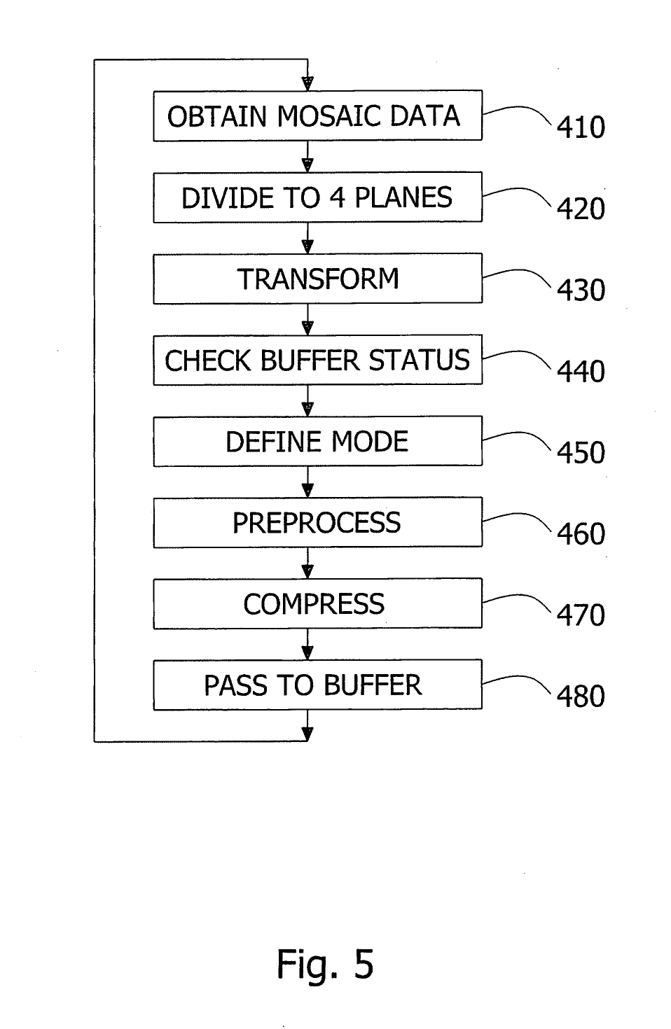

Reference is now made to FIG. 5, a flowchart describing a method controlling data flow through a buffer according to an embodiment of the present invention. In block 410 data, e.g. mosaic type data may be obtained. The mosaic type data may be, for example, a 256×256 pixel image. Other size data and data other than image data may be used, for example, a 512×512 pixel image may be obtained. In block 420 the mosaic image may be divided into its separate planes, for example four separate planes. Planes may be of any suitable shape, for example square, rectangle, etc. The planes may be separated as shown in FIG. 2B-2E according to the pixel arrangement shown in FIG. 2A. Other pixels arrangement may be used, for example, a mosaic pixel arrangement with two blue pixels for every red and green pixel or two red pixels for every blue and green pixel, or other colors may be included in the pixel arrangement. In other examples, there may be other pixel arrangements based on more or less than four pixels. Planes may not be actually separated, instead pointers may be defined or reference may be noted of where the pixels with the same colors may be located.

In block 430 a transformation may be implemented to transform, for example, the four planes to other dimensions and/or coordinates. In one embodiment of the present invention an exemplary transformation shown in FIG. 4 may be used. In other embodiments of the present invention, other dimensions may be defined and/or used, for example, one of the dimensions described herein or other known dimensions. In yet other embodiments of the present inventions, transformation need not be implemented, for example, transmission of data may be performed on the original coordinates. In block 440 the status of the buffer, e.g. vacancy or occupancy, or capacity level, of the buffer may be checked to prevent buffer overflow. If the occupancy or fullness is above a defined threshold, for example, more than half filled, more than two thirds filled, etc., a command to for example the compression module 100, may be sent from the buffer, for example to change the mode or operation, rate, etc, of compression so that overflow of the buffer may be avoided. The commands may for example be a software or hardware command and may be obtained from for example, circuitry within the in-vivo device 40 or for example from external commands transmitted to the in-vivo device 40. In other embodiments, commands to other units or other functionalities may be sent to for example change the mode of operation. In block 450 a new mode of operation may be defined. For example if the buffer may be filled above a defined max threshold a command to the compression module together with the pre-processing unit may be to increase the degree of compression and/or the pre-processing. In other embodiments, a command may be sent for example to an imager or other sensor to for example sample at a slower rate. Other suitable changes to the mode of operation may be implemented. For example if the buffer may be filled below, for example, a minimum threshold the degree of compression and or pre-processing may be, for example, reduced. Other suitable changes may be made. In block 460 pre-processing based on the mode defined may be performed. In block 470 the pre-processed data may be compressed and then passed on to the buffer (block 480).

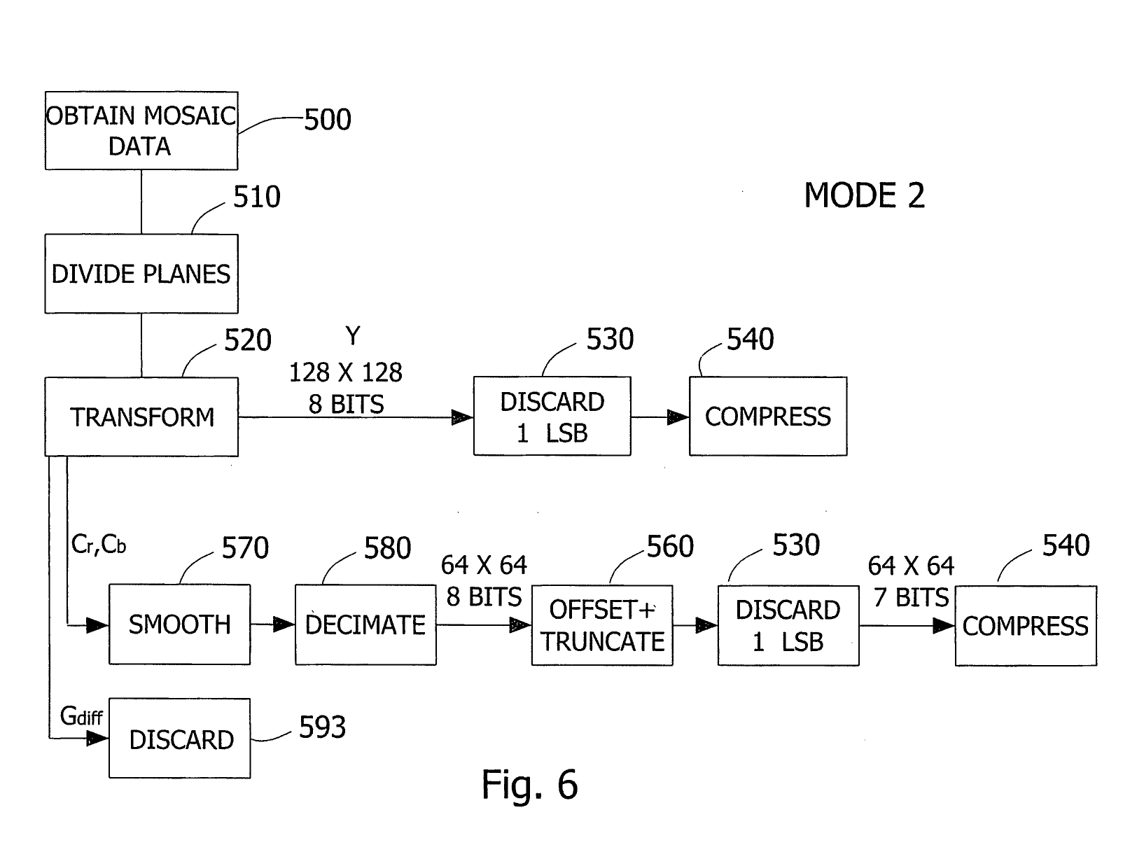

Reference is now made to FIG. 6 describing a method for compressing image data according to a defined mode, for example mode 2, in accordance with an embodiment of the present invention. In block 500 data, e.g. mosaic type data may be obtained. The mosaic type data may be, for example, a 256×256 pixel image. Other size data and data other than image data may be used, for example, a 512×512 pixel image may be obtained. In block 510 the mosaic image may be divided into its separate planes, for example four separate planes. The planes may be separated as shown in FIG. 2B-2E according to the pixel arrangement shown in FIG. 2A. Other pixels arrangement may be used, for example, a mosaic pixel arrangement with two blue pixels for every red and green pixel or two red pixels for every blue and green pixel, or other colors may be included in the pixel arrangement. In other examples, there may be other pixel arrangements based on more or less than four pixels. Planes may not be actually separated, instead pointers may be defined or reference may be noted of where the pixels with the same colors may be located. In block 520 a transformation may be implemented to transform, for example, the four planes to other dimensions and/or coordinates. In one embodiment of the present invention an exemplary transformation shown in FIG. 4 may be used. In other embodiments of the present invention, other dimensions may be defined and/or used, for example, one of the dimensions described herein or other known dimensions. In yet other embodiments of the present inventions, transformation need not be implemented, for example, compression may be performed on the original coordinates. In FIG. 4 the Y dimension may be representative of the intensity and in some embodiments may be regarded as containing a large part of the information. As such the pre-processing of the data in, for example, the Y dimension may be different from the other dimensions present. One of the methods that may be implemented to decrease the size of the data may be to discard one or more LSB(s), for example one LSB, as is shown in block 530, to for example decrease the size of one pixel of data from 8 to 7 bits. Other suitable methods may be employed to decrease the size of the data e.g. increase the resultant compression ratio.

In one embodiment of the present invention, the Y dimension may be compressed (block 540) even further using known lossless compression algorithms, for example, FELICS. Other suitable compression algorithms may be used. In one embodiment of the present invention, for one or more of the other dimensions, an alternate pre-processing may be implemented. In one example, the Cr and Cb planes may, for example, first be smoothed (block 570) using known methods. In one example, a smoothing filter with a 2×2 window may be implemented. In other examples other smoothing methods, e.g. linear and/or non-linear methods may be used. Subsequent to smoothing the data may be decimated (block 580) to reduce the size. For example, decimation may be used to reduce data in one or more dimensions, for example data having an original size of 128×128 bytes per image frame may be decimated to, for example, 64×64 bytes. In other examples, the data may be decimated to other suitable sizes. In block 560 data may be offset and truncated. One or more LSB(s) may be discarded (block 530) to decrease the size of the data before implementing a compression algorithm for example, FELICS. In other modes of operation and in other embodiments of the present invention, one or more blocks may not be implemented. For example, one or more of blocks 530, 560, 570, 580 may or may not be implemented. In other embodiments, other pre-processing may be implemented. In some embodiments of the present invention it may be desired to further reduce the processing power required to compress transmitted data. In one example, one or more of the dimensions defined by transform (520) may be discarded (block 593). Discarding a dimension of the defined data may serve to reduce the processing power required for compression and may increase the resultant compression ratio. In one example, the dimension defined by Gdiff may be, for example, discarded. In other examples, other dimensions or more than one dimension may be discarded. Other methods of increasing compression ratio, decreasing required processing power, and/or decreasing required memory capacity may be implemented.

Other modes of operation, for example modes, of compression and/or pre-processing may be used. Other modes of operation may be used. In some examples, the pre-processing defined for the Y dimension may be defined separately from, for example, the Cr, Cb dimension. In other embodiments other methods of defining modes of operation may be used. In this particular example, the Y dimension may be pre-processed less aggressively than for example the Cr and Cb dimensions and Gdiff dimension may be discarded. However, in other examples pre-processing of the Gdiff dimension may also be defined and the Y, Cr, and Cb dimensions may be pre-processed in alternate manners. In other embodiments mode of operation may be defined for operation other than pre-processing data or compressing data.

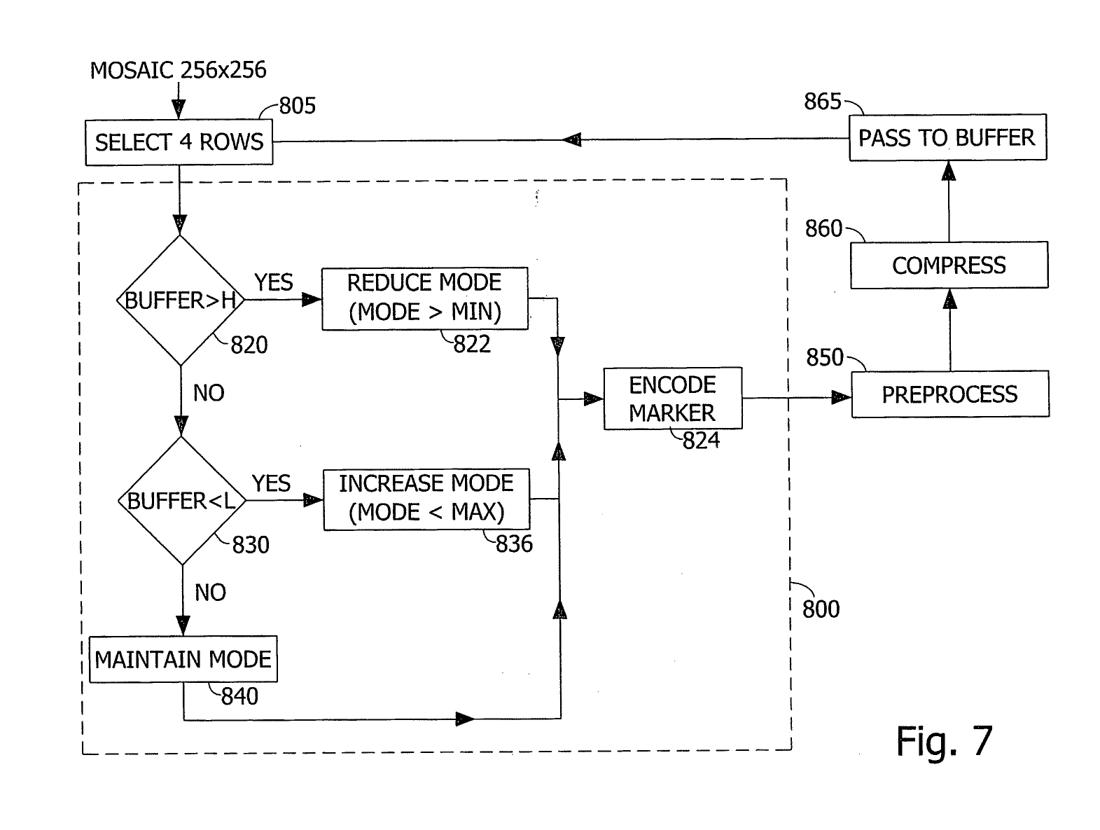

Reference is now made to FIG. 7 showing a flow chart describing a method for defining a mode or otherwise altering the operation of an in-vivo device, for example, altering a mode of compressing mosaic data to control the data flow through a buffer or other data storage unit according to an embodiment of the present invention. In block 805 four rows of mosaic data may be selected. In other examples more or less than four rows may be selected. For the first four rows of an image, a mode of operation may be predefined. A specific mode for dark images may be defined separately from regular images. In other examples other starting modes may be defined. For lines other than the first four lines, the occupancy or percent of capacity of the buffer 49 may be checked (block 820) before updating or selecting a mode of operation. If the buffer 49 is filled over a defined threshold, for example a threshold H, the current mode may be reduced by one (block 822), so that for example if the buffer 49 may be filling faster than the data may be transmitting, more aggressive pre-processing may be used, when exceeding the defined threshold, to reduce the data coming into the buffer 49 and to avoid possible overflow of the buffer 49. In some embodiments of the present invention, a change in mode may be denied so as to provide more stability, for example, if a previous change of mode was recently implemented a current change of mode may be denied. A marker may be encoded (block 824) to record the change in the operational mode, for example of the pre-processing/compression module. This information may be used by for example the decoder module 150 to decode the transmitted data. In some embodiments of the present invention, action may be taken for a case where there is an overflow or near overflow of data in the buffer. For example, in the case with overflow or near overflow, four lines of data may be skipped. In other embodiments, more or less than four lines of data may be skipped or other suitable action may be taken to control the overflow of data to the buffer. A marker may be encoded to indicate, for example, that lines were skipped and/or that there was an overflow of data. In another example, scanning of the lines may be frozen, for example on the analog to digital converter, until for example the overflow may be overcome. In other examples, the scanning may be frozen at other stages or may not be frozen. The time period of freezing may be variable or may be preset. For cases where the buffer 49 may be below a defined threshold, for example a defined threshold L (block 830), and the buffer 49 may not empty, the current mode of operation may be, for example increased by one, to for example, reduce the amount of pre-processing/compression preformed and/or required and the marker (block 824) may be encoded to, for example indicate the change for later use. If the buffer 49 may not be found to be empty, an empty code may be encoded into the marker to indicate that the buffer 49 may for example be transmitting null values. If the occupancy or percent of capacity of buffer 49 is between the H and L thresholds, the mode of operation may not be altered (block 840). Other suitable considerations and steps may be used to define the threshold of operation. In yet other embodiments of the present invention, the mode of operation may be constant and/or predefined. In some embodiments of the present invention, the mode of operation of the pre-processing and/or compression may be defined separately for dark images. In one example, dark images may not be pre-processed and/or compressed. In another embodiment, dark images may be pre-processed and/or compressed less as compared to other images. In other embodiments, the mode of pre-processing may alternate in a specified pattern for each slice (e.g. 4 lines) of dark image data processed. As such during decoding, dark image frames for each mode of operation may be reconstructed and subtracted from image frames processed with a similar mode of operation. In some embodiments of the present invention, thresholds H and L may be altered, for example, during the course of compressing an image frame. In one example, the threshold H may be reduced for the last few rows of an image frame so as not to create a lag in transmission toward the end of the image frame transmission. In other embodiments, H and L may be altered due to other conditions and/or due to a number of combined conditions that may include, for example, a current threshold value, current vacancy of buffer, current mode of operation, etc. Other suitable conditions may be used to alter H and L or other thresholds. Other changes in thresholds H and L may be made and other thresholds in addition to thresholds H and L may be used.

After defining the mode of operation (block 800), for example for pre-processing of data, pre-processing may be performed on the data (block 850) according to the mode defined, compression may be performed (block 860), for example, FELICS compression, and the data may be passed onto the buffer 49 (block 865) to await transmission. In some embodiments of the present invention, the mode of operation may be updated for every set of data selected. In other embodiments of the present invention, the mode of operation may be updated for every second, third or other number of sets of data selected. Data sent to the buffer 49 may include one or more markers to indicate a change in mode of operation and/or an empty buffer 49. In other embodiments of the present invention, the mode of operation may be defined by other parameters besides or in addition to pre-processing mode. The mode of operation may be defined by the frame capture rate of an imager, sampling rate of a sensor, or other operation of the in-vivo device 40. In other embodiments of the present invention, more or less steps may be defined to for example limit the number of modes that may be used or favor certain modes over others. In yet other embodiments of the present invention, other sets of steps may be used to define the mode of operation.

Compressed data may be transmitted to, for example, an external receiver 12, in portions. Compressed data that may be of variable size may pass through a buffer 49 before transmission. Once the portion defined for transmission may be filled, data accommodated in the buffer 49 may be transmitted. The data size of the defined portions may be the size of one or more images or may be the size of a few lines of data. The buffer 49 may provide for use of a constant bit rate transmitter for transmitting data of variable size. In one embodiment of the invention, compression data may be transmitted “on the fly” by transmitting portions equaling, for example, approximately four lines of pixel data while the buffer size may be in the order of magnitude of, for example, four lines of pixel data, e.g. 100 bytes to 2 kilobytes of data or less than 10 kilobytes of data, e.g. 340 bytes. Other suitable buffer sizes and transmission portions may be used. In some embodiments of the present invention, overflowing of the buffer 49 may be avoided by implementing a feed back mechanism between the buffer 49 and the pre-processing and/or compression algorithm as may be described herein. The feedback mechanism may control the pre-processing of the data based on the rate that the buffer 49 may be filling up. Other methods of transmitting and using buffers may be implemented, for example, using a buffer without feedback to compression and/or pre-processing algorithm.

Data transmitted may be received by an external receiver, for example receiver 12. Decoding and/or decompression may be performed on receiver 12 or on data processor 14 where data from the receiver may be subsequently downloaded. In an exemplary embodiment data decompression module 150 may be a microprocessor or other micro-computing device and may be part of the receiver 12. In alternate embodiments the functions of the data decompression (decoding) module 150 may be taken up by other structures and may be disposed in different parts of the system; for example, data decompression module 150 may be implemented in software and/or be part of data processor 14. The receiver 12 may receive compressed data without decompressing the data and store the compressed data in the receiver storage unit 16. The data may be later decompressed by, for example data processor 14.

Preferably, compression module 100 may be integral to imager 46. In other embodiments of the present invention, the data compression module 100 may be external to the imager 46 (e.g., integral to transmitter 41, a separate unit, etc.) and interface with the transmitter 41 to receive and compress image data; other units may provide other data to data compression module 100. In addition, the data compression module 100 may provide the transmitter 41 with information such as, for example, start or stop time for the transfer of image data from the data compression module 100 to the transmitter 41, the length or size of each block of such image data, and the rate of frame data transfer. The interface between the data compression module 100 and the transmitter 41 may be handled, for example, by the data compression module 100.

In alternate embodiments, the data exchanged between the data compression module 100 and the transmitter 41, or other units may be different, and in different forms. For example, size information need not be transferred. Furthermore, in embodiments having alternate arrangements of components, the interface and protocol between the various components may also differ. For example, in an embodiment where a data compression capability may be included in the transmitter 41 and the imager 46 may transfer uncompressed data to the transmitter 41, no start/stop or size information may be transferred.

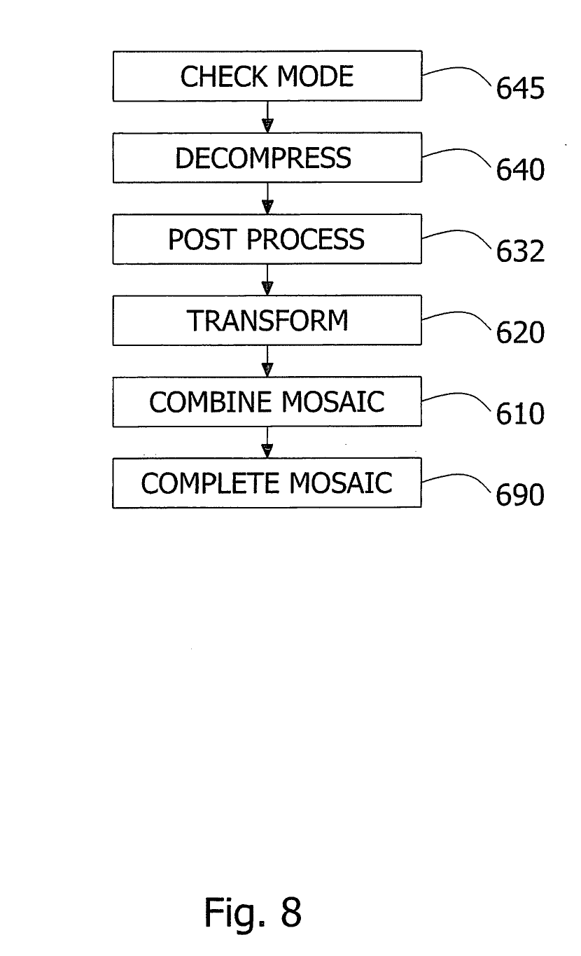

Reference is now made to FIG. 8 showing a flow chart describing a method for decompressing data according to an embodiment of the present invention. In block 645, the mode of operation may be checked, for example one or more markers may be read. In block 640, a decompression algorithm based on the compression algorithm used e.g. FELICS may be implemented. Based on the mode of operation, a suitable post-processing may be performed (block 632). In block 620, the data may be transformed to their original dimensions, e.g. (R, G1, G2, B), and the data may be combined to a mosaic frame (block 610). In block 690 the mosaic frame may be completed, by for example interpolation, or other methods so that, for example RGB data may be available for each pixel in the frame. Other suitable methods of decoding data may be used that may include more or less steps.

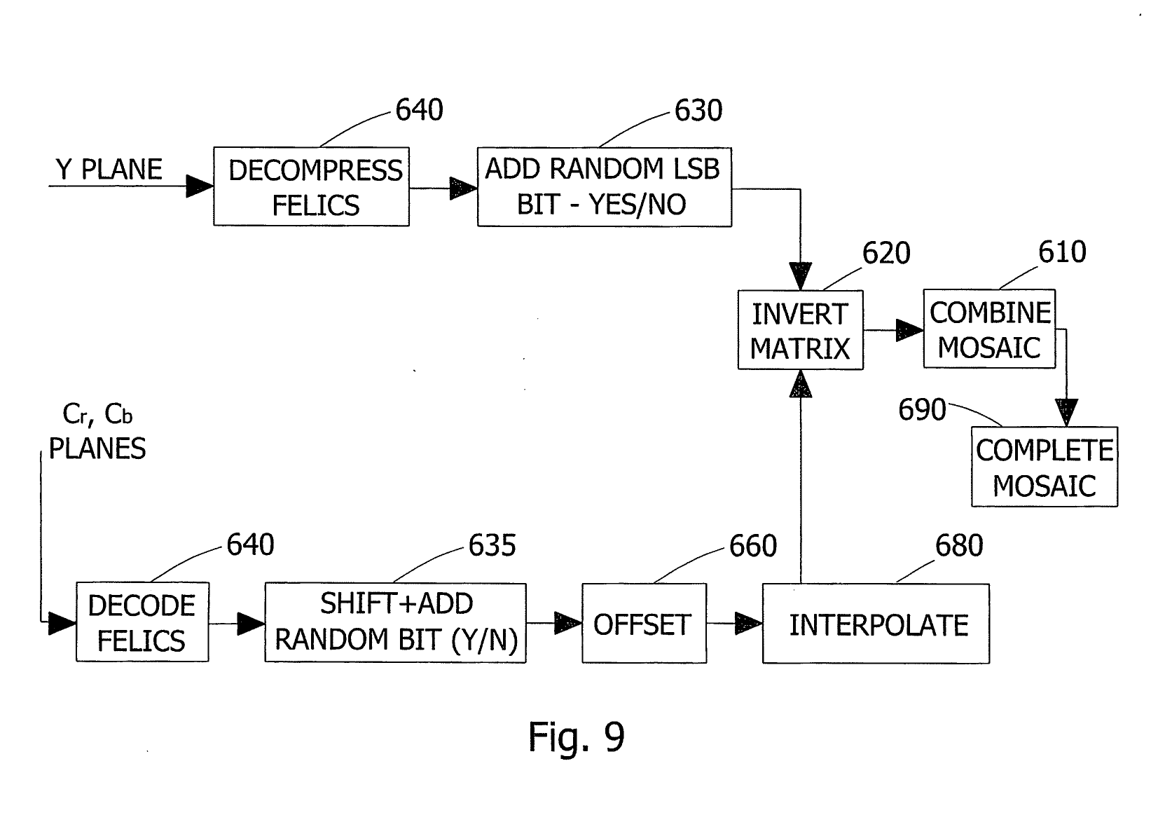

Reference is now made to FIG. 9 showing a flow chart describing a method for decompressing data that was compressed with a defined mode according to an embodiment of the present invention. Typically the method of decoding compressed data may be based on the method of compression, by generally reversing the steps taken for compression of the data, and pre-processing and/or post-processing of the data. For example, when using FELICS compression, the known decoding of FELICS may be implemented to decompress the compressed data (block 640). For reversing the pre-processing, the mode of operation may be read from one or more markers transmitted with the data. For example, for data pre-processed with mode 2, one random bit(s) of noise may be added (block 630) to the Y plane to replace the previously discarded LSB. The random bits may be generated by any of the known algorithms for generating random bits. In some embodiments of the present invention known pseudo-random bits may be used, for example, pseudo-random noise quantization using a dithering method. Other methods of producing pseudo-random bits may be used and dithering may be used during encoding. In still other embodiments of the present invention bits generated from a suitable algorithm may be used. Decoding of the other dimensions and/or planes, e.g. [Cr, Cb], may be accomplished in a similar manner. Decompression based on the implemented compression algorithm, for example FELICS (block 640), may be performed on each of the dimensions. The one LSB that may have been discarded may be replaced (block 635) as described herein. Offsetting may be reversed (block 660) and interpolation (block 680), for example, linear interpolation may be performed to restore, for example, decimated data to their original size. In other embodiments of the present invention, Gdiff, may not have been transmitted and as such not decoded. In block 620, the data from all the dimensions may be transformed to, for example, their original coordinates, for example to RG1G2B coordinates. In block 610 the image may be restored, e.g. the individual color planes may be combined onto one plane (FIG. 2A), and filled (block 690) to a true RGB image by known interpolation and/or other suitable methods so that for example, there may be an RGB value for each pixel. Compression and decoding of the mosaic image as opposed to the true RGB image may serve to minimize and/or reduce the processing power, rate, and memory required to compress image data. Subsequent to decoding, dark reference image frames may be subtracted from corresponding images if required.

Embodiments of the present invention may include apparatuses for performing the operations herein. Such apparatuses may be specially constructed for the desired purposes (e.g., a “computer on a chip” or an ASIC), or may include general purpose computers selectively activated or reconfigured by a computer program stored in the computers. Such computer programs may be stored in a computer readable storage medium, such as, but is not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions.

The processes presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems appears from the description herein. In addition, embodiments of the present invention are not described with reference to any particular programming language. It will be appreciated that a variety of programming languages may be used to implement the teachings of the invention as described herein.

Unless specifically stated otherwise, as apparent from the discussions herein, it is appreciated that throughout the specification discussions utilizing terms such as “processing”, “computing”, “calculating”, “determining”, or the like, typically refer to the action and/or processes of a computer or computing system, or similar electronic computing device (e.g., a “computer on a chip” or ASIC), that manipulate and/or transform data represented as physical, such as electronic, quantities within the computing system's registers and/or memories into other data similarly represented as physical quantities within the computing system's memories, registers or other such information storage, transmission or display devices.

It will be appreciated by persons skilled in the art that the present invention is not limited to what has been particularly shown and described hereinabove. Rather the scope of the present invention is defined only by the claims, which follow:

1. An in-vivo imaging device for transmitting data comprising:
an imager;
a buffer to store image data wherein the buffer has a capacity that is smaller than a frame of image data; and
a transmitter.

2. The in-vivo device of claim 1, wherein the imager comprises a CMOS.

3. The in-vivo device of claim 1 wherein the image data is mosaic image data.

4. The in-vivo device of claim 1 comprising a data compression module.

5. The in-vivo device of claim 4 wherein the compression module has more than one mode of operation.

6. The in-vivo device of claim 1 wherein the buffer has a capacity of between 100 bytes and 2 kilobyte.

7. The in-vivo device of claim 1 wherein a mode of operation of the in-vivo device is updated based on occupancy of the buffer.

8. The in-vivo device of claim 1 wherein the buffer is integral to the imager.

9. The in-vivo device according to claim 1 wherein the buffer is integral to the transmitter.

10. The in-vivo device of claim 1 wherein the transmitter is an RF transmitter.

11. The in-vivo device of claim 1 wherein the transmitter is a constant bit rate transmitter.

12. An in-vivo device for transmitting in-vivo data comprising:
a sensor to sample in-vivo data;
a buffer to store data obtained at a variable bit rate; and
a transmitter.

13. The in-vivo device according to claim 12 wherein the sensor is an imager.

14. The in-vivo device according to claim 12 wherein the data is image data.

15. The in-vivo device according to claim 12 wherein the data is compressed image data.

16. The in-vivo device according to claim 12 wherein the capacity of the buffer is less than 20 kilobytes.

17. The in-vivo device according to claim 12 wherein the transmitter is to transmit data from the buffer.

18. The in-vivo device according to claim 12 wherein the transmitter is a constant bit rate transmitter.

19. A method for transmitting data from an in-vivo device, the method comprising:
obtaining data at a variable bit rate;
collecting in-vivo data;
passing the in-vivo data to an in-vivo buffer;
transmitting data from the buffer at a constant bit rate; and
changing mode of operation of the in-vivo device based on occupancy of the buffer.

20. The method according to claim 19 wherein the in-vivo data comprises image data.

21. The method according to claim 19 wherein the constant bit rate is 1 to 10 megabits/second.

22. The method according to claim 19 wherein the mode of operation comprises a mode of processing data.

23. The method according to claim 19 comprising encoding a marker indicating the mode of operation.

24. The method according to claim 19 comprising encoding a marker to indicate transmission of null values.

25. The method according to claim 19 comprising encoding a marker to indicate overflow of the buffer.

26. The method according to claim 19 comprising reducing the mode of operation when the buffer occupancy exceeds a given threshold.

27. The method according to claim 19 comprising increasing the mode of operation when the buffer occupancy is below a given threshold.

28. The method according to claim 19 wherein the step of passing data to a buffer comprises passing data that is less than an image frame.
